(19) United States
(12) Patent Application Publication
(10) Pub. No.: US 2009/0012539 A1
(43) Pub. Date: Jan. 8, 2009

(54) Surgical clamping instruments and methods

(76) Inventors: Gary Louis Zohman, Calabasas, CA (US); J. Michael Kabo, Granada Hills, CA (US)

Correspondence Address: LAW OFFICE OF ALAN W. CANNON, 942 MESA OAK COURT, SUNNYVALE, CA, 94086, US

(21) Appl. No.: 11/825,182

(22) Filed: Jul. 3, 2007

(51) Int. Cl. A61B 17/08; A61B 17/30; A61B 17/00

(52) U.S. Cl. 606/151; 606/208

(57) ABSTRACT

Surgical clamping instruments include a distal portion having distal tips configured to be driven to provide clamping force to multiple tissue pieces; a proximal portion including a compression actuator configured for operation by a user to drive the distal tips to apply compression force to the tissue pieces, and a shear actuator configured for operation by the user to drive the distal tips to apply shear force to the tissue pieces; and an intermediate portion interconnecting the proximal and distal portions. A method of joining tissues pieces together includes applying first and second force applicators to first and second surfaces of first and second tissue pieces, wherein the first and second surfaces oppose third and fourth surfaces of the first and second tissue pieces to be joined, respectively; applying compressive force, via the first and second force applicators, to drive the first and second pieces together; and applying shear force, via said first and second force applicators, to accurately align the third and fourth surfaces, so that the third and fourth surfaces are contacted to one another in a desired fit pattern.

### FIELD OF THE INVENTION

The present invention relates broadly to clamping instruments used in orthopedic surgery and methods of using the same during such surgery.

### BACKGROUND OF THE INVENTION

A reduction clamp or tenaculum is a simple hardware tool used by orthopedic surgeons to hold pieces of bone together during surgery. This tool has essentially the same design as an ordinary clamp used by a carpenter and sold in a hardware store. The main difference is that clamps used for bone usually have sharp points and close with precision. Like ordinary clamps, there is a locking mechanism so that compression can be maintained between the points of the clamp.

U.S. Pat. No. 4,475,544 to Reis discloses a forceps-type bone clamp having distal pointed ends that are turned inwardly toward one another. A ratcheting mechanism is provided near the proximal end of the clamp, between scissors-type handles, so that a clamping force can be maintained. This type of design allows for the creation of a compressive force that is always directed between the points of the clamp. The ease of use of this instrument depends entirely on the orientation of the two surfaces being compressed. If the clamp can be positioned perpendicular to the surfaces, then pure compression will occur. If, on the other hand, the clamp is not perpendicular to the plane of desired compression, then the clamp will cause shear as well as compression. This can be problematic, because it is generally intended to clamp the broken pieces back together by driving them in a straight line of compression toward one another. The shearing force can drive the bone pieces out of alignment, so that when the bone pieces make contact as a result of the combined shear and compression forces, the bones are out of alignment relative to the positions that the orthopedic surgeon intended to join them in. Proper alignment of the bones is extremely important in order to minimize occurrences of nonunion, among other issues.

In order to correct for the misalignment caused by the shear forces, a second (and maybe even a third or more) clamp(s) are required to provided a compression force to drive the misaligned bone pieces toward the intended positions where they are properly aligned. This becomes critical during fracture surgery. It is often difficult to fit one clamp into the operative field, let alone two. Multiple clamps protruding from the surgical wound make access difficult for the drills and screwdrivers that must be inserted in order to repair the fracture.

Thus, there is a need for clamping instruments and methods that can produce a force vector that is not limited to the line connecting the points on the bone pieces to be clamped, i.e., that is not limited to providing compressive force along a single line, such as what the clamps of the prior art are limited to.

### SUMMARY OF THE INVENTION

The present invention provides clamping instruments including: a distal portion having distal tips configured to be driven to provide clamping force to multiple tissue pieces; a proximal portion including a compression actuator configured for operation by a user to drive the distal tips to apply compression force to the tissue pieces, and a shear actuator configured for operation by the user to drive the distal tips to apply shear force to the tissue pieces; and an intermediate portion interconnecting the proximal and distal portions.

In at least one embodiment, a relative position locking mechanism is configured to temporarily maintain the distal tips in constant relative positions to apply a constant amount of compression.

In at least one embodiment, a relative position locking mechanism configured to temporarily maintain the distal tips in constant relative positions to apply a constant amount of shear.

In at least one embodiment, the instrument comprises a bone clamp.

In at least one embodiment, the tissue pieces are fractured pieces of a bone.

In at least one embodiment, the distal portion comprises a pair of elongate shafts, each provided with one of the distal tips at a distal end thereof.

In at least one embodiment, the distal tips are directed inwardly toward one another.

In at least one embodiment, the intermediate portion comprises first and second supports, a first of the distal tips being connected to the first support and a second of the distal tips being connected to the second support, the shear actuator being operable to drive a relative tilting action between the first and second supports to generate the shear force in the distal tips.

In at least one embodiment, the intermediate portion comprises first and second supports, a first of the distal tips being connected to the first support and a second of the distal tips being connected to the second support, the compression actuator being operable to drive a relative rotation action between the first and second supports to generate the compression force in the distal tips by driving the distal tips toward one another.

In at least one embodiment, the intermediate portion comprises first and second supports, the first and second supports being connected via a joint that permits relative rotation between the first and second supports and relative tilting between the first and second supports.

In at least one embodiment, the joint comprises a swivel bearing.

In at least one embodiment, the intermediate portion comprises a shear driver pivotally fixed to the intermediate portion and configured to provide a driving force against one of the first and second supports, when driven by the shear actuator, to tilt one of the first and second supports relative to the other of the first and second supports.

A method of joining tissues pieces together is provided, including the steps of: applying first and second force applicators to first and second surfaces of first and second tissue pieces, wherein the first and second surfaces oppose third and fourth surfaces of the first and second tissue pieces to be joined, respectively; applying compressive force, via the first and second force applicators, to drive the first and second pieces together; and applying shear force, via the first and second force applicators, to accurately align the third and fourth surfaces, so that the third and fourth surfaces are contacted to one another in a desired fit pattern.

The first and second force applicators can be provided as distal tips of a single clamping instrument according to the present invention.

In at least one embodiment, the tissue pieces are bone pieces, and the first and second force applicators are portions of a single bone clamping instrument.

In at least one embodiment, the application of compressive force is performed first, followed by fine tuning of alignment of the pieces by the application of shear forces.

In at least one embodiment, the compressive force and the shear force are applied simultaneously.

In at least one embodiment, the application of compressive force and the application of shear force are alternatively and iteratively adjusted to accurately align the tissue pieces and clamp them together to contact the third and fourth surfaces together in accurate alignment.

A method of accurately clamping fractured bone pieces together with a single bone clamp is provided, including the steps of: applying first and second distal tips of the bone clamp to first and second surfaces of first and second bone pieces, wherein the first and second surfaces oppose third and fourth fracture surfaces of the first and second bone pieces to be joined, respectively; applying compressive force, via the first and second distal tips, to drive the first and second pieces together; and applying shear force, via the first and second distal tips, to accurately align the third and fourth fracture surfaces, so that the third and fourth fracture surfaces are contacted to one another in a desired fit pattern.

The application of compressive force and the application of shear force can be independently controlled via a compression actuator and a shear actuator, respectively, provided in a proximal portion of the bone clamp.

These and other features of the invention will become apparent to those persons skilled in the art upon reading the details of the instruments and methods as more fully described below.

### BRIEF DESCRIPTION OF THE DRAWINGS

FIG. 1 illustrates a prior art forceps-type bone clamp.

FIG. 2A schematically illustrates a situation in which the distal tips of a clamp are capable of being positioned to apply pure compressive force perpendicular to surfaces of two pieces of bone to be rejoined during surgery.

FIG. 2B schematically illustrates a situation in which the distal tips of the clamp of FIG. 1 are not capable of being positioned to apply compressive force perpendicular to surfaces of two pieces of bone to be rejoined during surgery.

FIG. 2C illustrates movements of the bone pieces 1, 2 into misalignment, as a result of application of force in a manner as described with regard to FIG. 2B.

FIG. 2D illustrates compressive forces applied by a first clamp at one location, and compressive forces applied by a second clamp at a second location, to counter the shear forces resulting from the compressive forces applied at the first location.

FIG. 3 schematically illustrates application of forces by an instrument according to the present invention to accurately compress the surfaces together in a situation where the fracture is oriented like that described with regard to FIGS. 2B-2D.

FIG. 4A illustrates a plan view of an embodiment of an instrument according to the present invention.

FIG. 4B illustrates a perspective view of the instrument of FIG. 4A, showing the opposite side of the instrument.

FIG. 5 is an exploded view of the instrument shown in FIG. 4B.

FIG. 6 is a partial, enlarged side view of the instrument of FIG. 5 showing the assembled components of the intermediate portion.

FIG. 7A is a partial view of an alternative embodiment according to the present invention.

FIG. 7B is an enlarged partial view of FIG. 7A.

### DETAILED DESCRIPTION OF THE INVENTION

Before the present instruments and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a handle” includes a plurality of such handles and reference to “the arm” includes reference to one or more arms and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

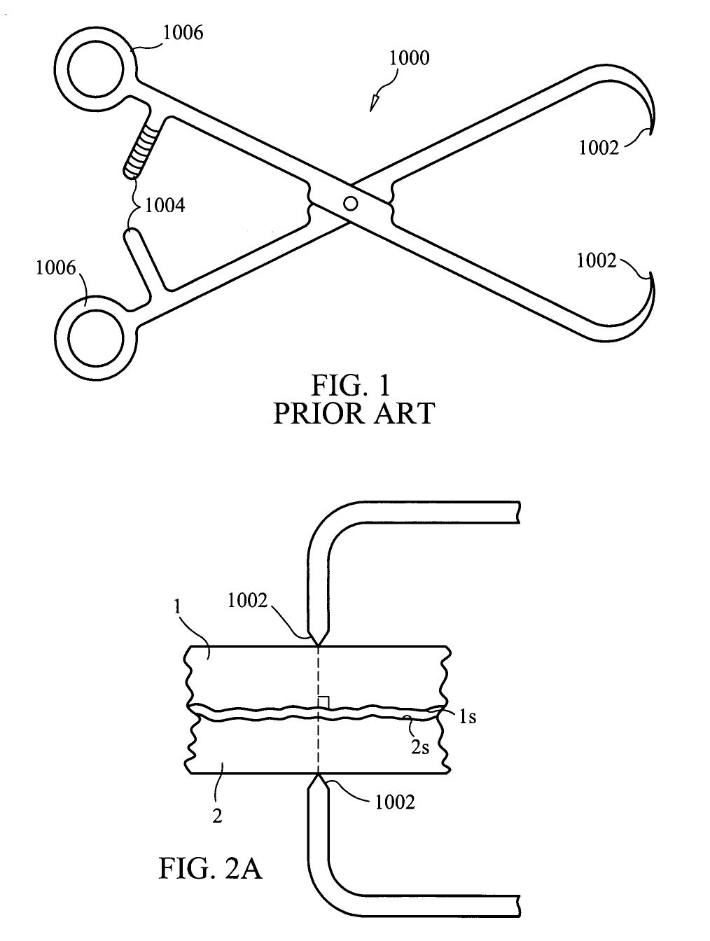

FIG. 1 illustrates a prior art forceps-type bone clamp 1000 having distal pointed ends 1002 that are turned inwardly toward one another. A ratcheting mechanism 1004 is provided near the proximal end of the clamp, between scissors-type handles 1006, so that a clamping force can be maintained. This type of design allows for the creation of a compressive force that is always directed between the points 1002 of the clamp 1000 along a single line. The ease of use of this instrument depends entirely on the orientation of the two surfaces being compressed. If the clamp can be positioned perpendicular to the surfaces, then pure compression will occur. If, on the other hand, the clamp is not perpendicular to the plane of desired compression, then the clamp will cause shear as well as compression. This can be problematic, because it is generally intended to clamp the broken pieces back together by driving them in a straight line of compression toward one another. The shearing force can drive the bone pieces out of alignment, so that when the bone pieces make contact as a result of the combined shear and compression forces, the bones are out of alignment relative to the positions that the orthopedic surgeon intended to join them in. Proper alignment of the bones is extremely important in order to minimize occurrences of nonunion, among other issues.

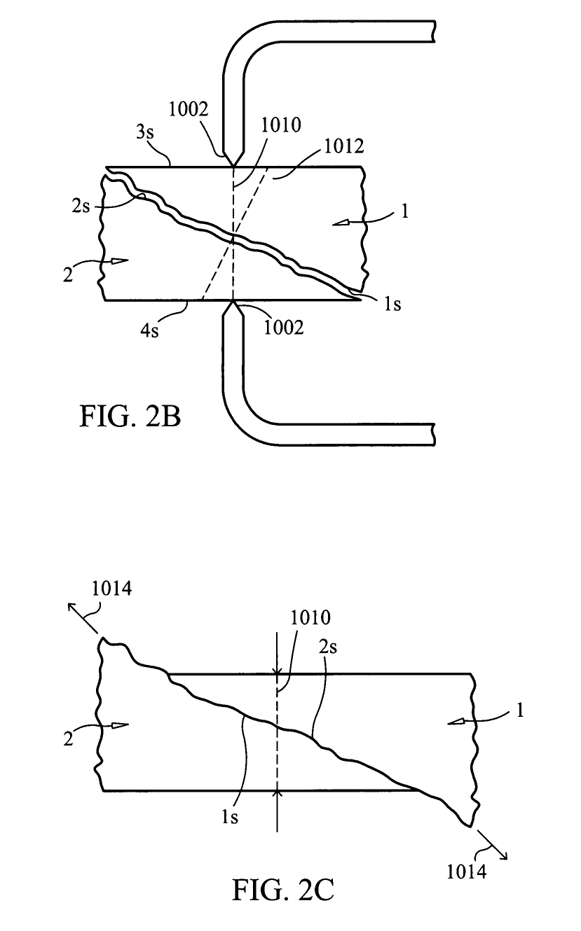

FIG. 2A schematically illustrates a situation in which the distal tips 1002 of clamp 1000 are capable of being positioned to apply compressive force perpendicular to surfaces 1s, 2s of two pieces of bone 1,2 to be rejoined during surgery. This situation is ideal for the application of clamping forces by clamp 1000 as forces can be applied along a straight line that is perpendicular to the surfaces 1s, 2s as shown. Accordingly, surfaces 1s, 2s can be clamped together without misalignment, as the surfaces are driven together along the direction of the line shown in dashed lines in FIG. 2A. Unfortunately, this situation is not very common, as the situation much more often presents with the fracture surfaces 1s, 2s of the bones being oriented such that the tips 1002 can not be aligned perpendicular to the surfaces.

FIG. 2B schematically illustrates a situation in which the distal tips 1002 of clamp 1000 are not capable of being positioned to apply compressive force against surfaces 3s, 4s to apply forces perpendicular to surfaces 1s, 2s of two pieces of bone 1,2 to be rejoined during surgery. Accordingly, when distal tips 1002 are contacted to the surfaces 3s, 4s of bone pieces 1, 2 as illustrated in FIG. 2B, and compressive force is applied between the two tips 1002 along the direction of dashed line 1010, the resultant force components from this action include a compression in the direction of line 1012, as well as shear force in the direction of a line perpendicular to line 1012. FIG. 2C illustrates the movements of the bone pieces 1, 2 into misalignment, as a result of application of force in a manner as described with regard to FIG. 2B. Specifically, application of compression forces by the clamp tips 1002 along the line 1010, as indicated by the arrows aligned with line 1010, drives pieces 1, 2 in compression into contact with one another, but also drives pieces 1,2 in the directions of the shear force arrows 1014. This results in the surfaces 1s, 2s becoming misaligned as they contact one another, such that the bone surfaces are not contacted back together in the same relative orientations that they were in prior to the bone breaking, when the surfaces 1s, 2s were created.

Since a goal of the surgery is to reconstruct the bone as close to possible as it was prior to the fracture, the result in FIG. 2C is unacceptable, and the bone pieces 1, 2 must be repositioned prior to be fixed together (e.g., such as by screws, pins, plates, or some other technique known in the art). In order to correct this situation, the shear forces need to be negated or neutralized, and this is typically done by applying one or more additional clamps at different orientations. For example, FIG. 2D illustrates compressive forces applied by a first clamp 1000 via clamp tips 1002 at arrows 1016, 1016 to apply force in the same manner as described above with regard to FIGS. 2B-2C. Additionally, compressive forces are applied by a second clamp 1000 via tips 1002 in the locations indicated by arrows 1018 to counter the shear forces resulting from the compressive forces applied at arrows 1016, 1016. Sometimes a third or more clamps 1000 are required to be applied to correct the forces so as to fit the bone pieces 1, 2 back together correctly under clamping. As noted above, this can be problematic because it is often difficult to fit one clamp into the operative field, making it much more difficult, or impossible to fit a second clamp or more. Additionally, the requirement to place multiple clamps takes more time, increasing the cost of the surgical procedure and potentially also increasing risks. Further, the presences of multiple clamps protruding from a surgical wound makes access by other instruments more difficult, e.g., drills, screwdrivers, etc.

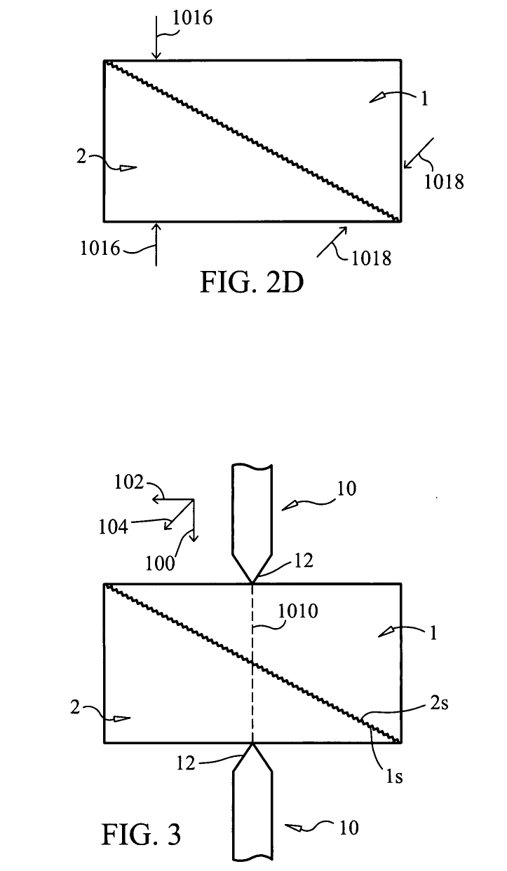

The present invention provides a single instrument that is capable of applying a compressive force between two points, like that described with regard to clamp 100 above, while it can additionally apply a shear force so as to negate the need for one or more additional clamps in a situation such as that described above with regard to FIGS. 2B-2D. Instruments described herein are particularly useful in orthopedic surgery, for clamping bone to bone, or implants (such as metal or ceramic plates, or the like) to bone, although these instruments may be useful for clamping other tissues as well. FIG. 3 schematically illustrates application of forces by an instrument according to the present invention to accurately compress the surfaces 1s, 2s together in a situation where the fracture is oriented like that described above with regard to FIGS. 2B-2D. In this case, instrument 10 is arranged so that distal tips 12 contact the bone pieces 1 and 2 in the same locations as described above with regard to FIG. 2B. Instrument 10 can be operated to apply compressive forces between the two tips 12 in a direction along the line 1010 in the same manner as clamp 1000, which is adequate if the surfaces 1s, 2s are perpendicular to line 1010, as noted above. However, in situations such as the one shown in FIG. 3, instrument 10 can be operated to additionally apply a shear force between tips 12, so that the combined force vector compresses the bone surfaces 1s, 2s together in the intended relative positions, without shearing the pieces away from one another. Arrow 100 indicates the direction of the compressive force applied by the upper tip 12 relative to the second tip 12 and arrow 102 indicates the direction of the perpendicular force applied by the upper tip 12 relative to the second tip 12. Arrow 104 indicates the combine force, i.e., resultant force vector 104, which is perpendicular to the surfaces 1s, 2s.

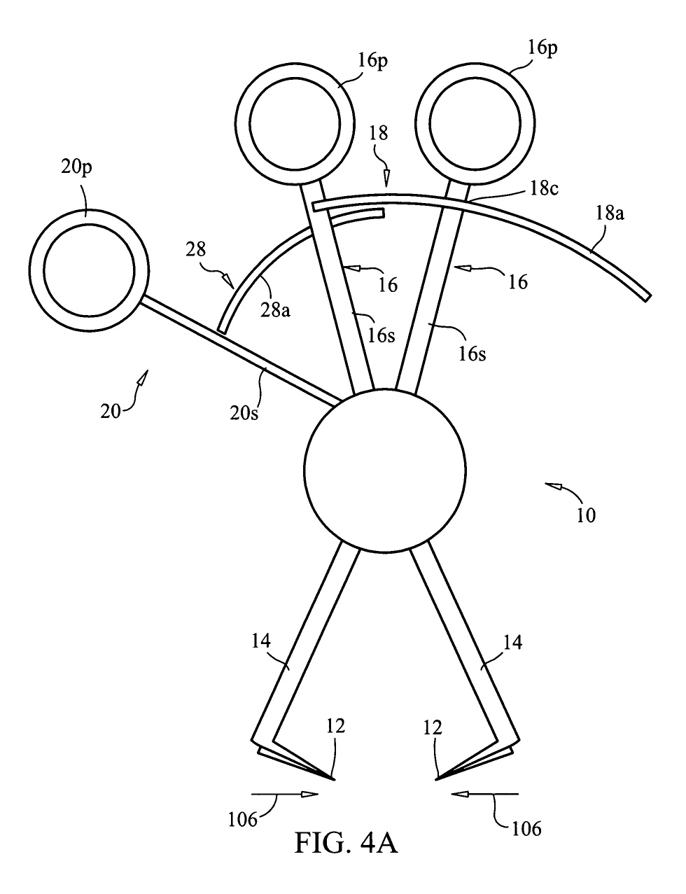

FIG. 4A illustrates a plan view of an embodiment of an instrument 10 according to the present invention, and FIG. 4B illustrates a perspective view showing the opposite side of the instrument 10 of FIG. 4A. Instrument 10 includes first and second elongated shafts 14 at a distal portion thereof configured to be rotated or pivoted toward or away from one another. Distal tips 12 are directly inwardly toward one another, so that when instrument 10 is operated solely in compression, tips 12 can be driven toward one another along a force line that interconnects the aligned tips 12 (e.g., line 1010 in FIG. 3) Force arrows 106 illustrate the direction of application of the compressive forces described.

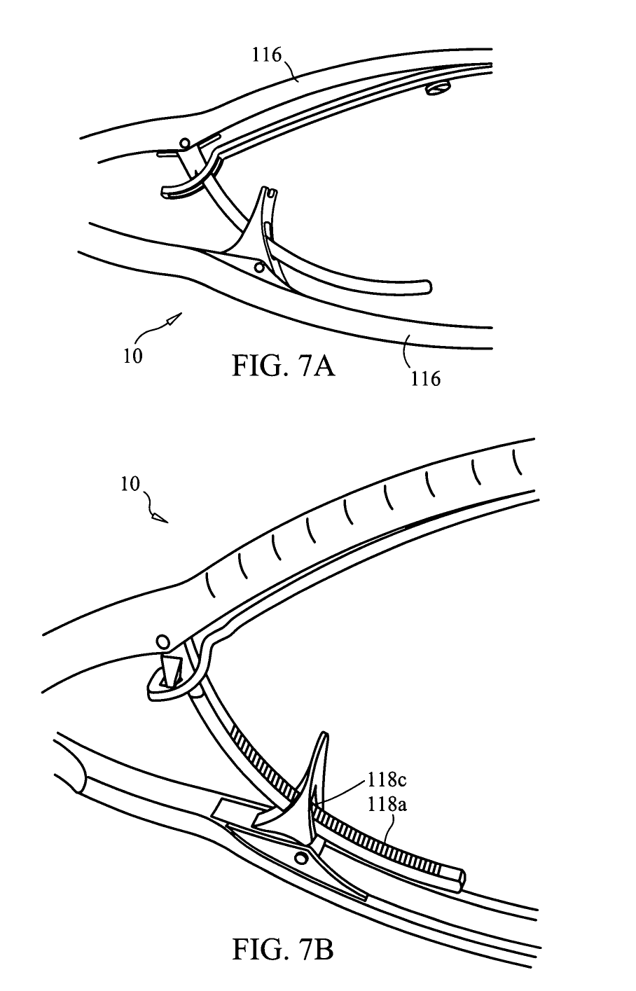

Compression actuators 16 are included in a proximal portion of instrument 10 and are configured to be manually operated by a user (e.g., surgeon or other medical personnel) to apply compression via tips 12. In the example of FIG. 4A, compression actuators 16 each include an elongated shaft 16s and a loop 16p that facilitates ease of operation, by allowing the user to insert a finger or thumb through each loop 16p. However, it is noted that the present invention is not limited to shaft and loop type compression actuators 16, as compression actuators may take other forms. For example, instead of loops 16p, curved handles, such as concave shaped handles may be provided. Further alternatively, convex shaped handles 116 may be provided to replace the shafts 16 and loops 16p of the device 10 shown in FIG. 4A, as illustrated in the partial view of an alternative device embodiment 10 shown in FIG. 7A. Further alternatively, shafts 16s can be interconnected by a threaded, spring-loaded rod, such that threading a nut down on the rod drives shafts 16s closer together, while back-threading allows the spring to drive the shafts 16s further apart. Many other configurations may be substituted to perform the compression driving function, as would be readily apparent to one of ordinary skill in the mechanical arts.

A relative position locking mechanism 18 is provided on compression actuators 16 to lock compression actuators in their current positions relative to one another until such time as the user decides to alter the relative positions. In the example shown in FIGS. 4A and 4B, relative positioning locking mechanism 18 comprises a ratcheting mechanism in which a curved, toothed, ratchet arm 18a extends transversely away from the shaft 16s that it is connected to, towards and beyond the other shaft 16s. The other shaft includes a ratchet catch 18c mounted thereon, in a location to interact with the teeth on ratchet arm 18a to form a temporary lock therewith. The ratchet arm 18a and catch 18c engage with one another to lock the positions of arms 16s relative to one another, which is useful to maintain a constant amount of compression between tips 12 during use of instrument 10. This locking mechanism can be released by separating the teeth of ratchet arm 18a from catch 18c, by moving the ratchet arm 18a and/or the catch 18c perpendicularly to the direction that they move relative to one another during increasing or decreasing the distance between arms 16s. Relative position locking mechanism 18 is not limited to the ratchet mechanism described above and shown in FIGS. 4A-4B, as other types of relative position locking mechanisms may be substituted. For example, in the case where shafts 16s are interconnected by a threaded, spring-loaded rod, the nut and threaded rod also function as a relative position locking mechanism. FIG. 7B is an enlarged partial view of FIG. 7A that illustrates an alternative ratchet and locking arrangement 118a, 118c. Many other configurations may be substituted to perform the relative position locking function, as would be readily apparent to one of ordinary skill in the mechanical arts.

Shear actuator 20 is included in a proximal portion of instrument 10 and is configured to be manually operated by a user (e.g., surgeon or other medical personnel) to apply shear via tips 12. In the example of FIGS. 4A-4B, shear actuator 20 includes an elongated shaft 20s and a loop 20p that facilitates ease of operation, by allowing the user to insert a finger or thumb through loop 20p to facilitate non-slip operation. However, it is noted that the present invention is not limited to shaft and loop type shear actuator 20, as shear actuator 20 may take other forms. For example, instead of loop 20p, a curved handle or other handle shaped to facilitate manipulation by a user may be provided. Further alternatively, shafts 20s can be interconnected to adjacent shaft 16s by a threaded, spring-loaded rod, such that threading a nut down on the rod drives shaft 20s toward shaft 16s, while back-threading allows the spring to drive the shafts 20s away from shaft 16s. Many other configurations may be substituted to perform the shear actuation or driving function, as would be readily apparent to one of ordinary skill in the mechanical arts.

A relative position locking mechanism 28 is provided to lock a relative position of shear actuator 20 relative to the adjacent compression actuator shaft 16s, until such time as the user decides to alter the relative position of the shear actuator 20s. In the example shown in FIGS. 4A and 4B, relative positioning locking mechanism 28 comprises a ratcheting mechanism in which a curved, toothed, ratchet arm 28a extends transversely away from the shaft 20s that it is connected to, towards and beyond the adjacent shaft 16s. The adjacent shaft 16s includes a ratchet catch 28c mounted thereon, in a location to interact with the teeth on ratchet arm 28a to form a temporary lock therewith. The ratchet arm 28a and catch 28c engage with one another to lock the position of arm 20a relative to the adjacent arm 16s, which is useful to maintain a constant amount of shear between tips 12 during use of instrument 10. This locking mechanism can be released by separating the teeth of ratchet arm 28a from catch 28c, by moving the ratchet arm 28a and/or the catch 28c perpendicularly to the direction that they move relative to one another during increasing or decreasing the distance between arm 20s and adjacent arm 16s. Relative position locking mechanism 28 is not limited to the ratchet mechanism described above and shown in FIGS. 4A-4B, as other types of relative position locking mechanisms may be substituted. For example, in the case where shaft 20s and adjacent shaft 16s are interconnected by a threaded, spring-loaded rod, the nut and threaded rod also function as a relative position locking mechanism. Many other configurations may be substituted to perform the relative position locking function, as would be readily apparent to one of ordinary skill in the mechanical arts.

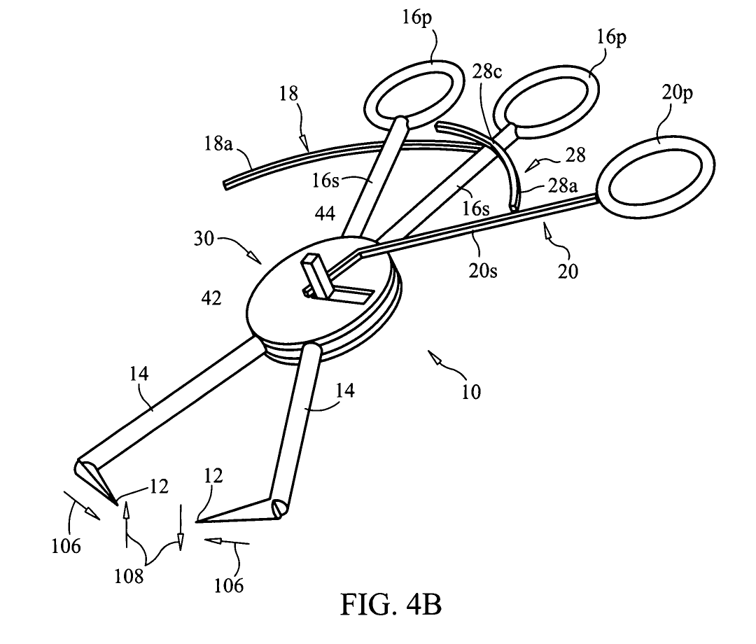

FIG. 5 is an exploded view of instrument 10 in the orientation shown in FIG. 4B, and is provided to better illustrate the shear mechanism in the intermediate portion 30 of instrument 10 as well as the connections of the shafts to the intermediate portion. Intermediate portion 30 includes first and second support members 32 and 34, respectively, interconnected to one another to allow rotation of each support member 32,34 about transverse axis 36 and relative to each other, to effect compression, for example, as well as tilting of the support members 32,34 relative to one another to effect shear, for example. A first of the pair of shafts 16s and a first of the pair of shafts 14 are rigidly connected to first support member 32 to oppose one another so that movement of the first shaft 16s in one direction rotates support member 32 to drive the first shaft 14 in the opposite direction. Likewise, the second of the pair of shafts 16s and the second of the pair of shafts 14 are rigidly connected to second support member 34 to oppose one another so that movement of the first shaft 16s in one direction rotates support member 32 to drive the first shaft 14 in the opposite direction.

A swivel bearing 36 interconnects first and second support members 32, 34 to allow relative rotational movements as well as relative tilting movements between the support members 32, 34. A securing mechanism, such as a pin, rivet, or nut and bolt, or the like are provided through support members 32,34 and swivel bearing 36 to secure the components together. Clearance is provided for the holes in support members 32,34 and swivel bearing 36, so that they have larger inside diameters than the outside diameter of the securing mechanism that passes therethrough, to permit relative motion of support members 32,34 about swivel bearing 36, including both rotational and tilting movements.

A shear driver 38 (e.g., lever, or the like) is partially inserted through a slot 40 in first support member 32. Shear driver 38 is also hingedly or pivotally connected to support member 32 at hinge or pivot point 42 via a hinge or pivot joint. Shaft 20s is hingedly or pivotally connected to an external surface of support member 32 at hinge or pivot point 44. Upon actuation of shear actuator 20 by moving it in a direction toward the adjacent shaft 16s, this causes the distal end portion of shaft 20s to drive against a portion of shear driver 38 that extends out of support member and is located on one side of joint 42. This rotates shear driver, thereby driving the portion of shear driver on the other side of joint 42 against support member 34, thereby tilting support member 34 relative to support member 32 and causing a shearing force to be delivered through distal tips 12 when they are engaged with surfaces, such as bone pieces, for example. In FIG. 5, the action just described causes the right side of support member to move downward (away) from the right side of support member 32, thus effecting shear forces in the directions of arrows 108 in FIG. 5. Upon release of the relative position locking mechanism 28 and movement of arm 20s away from the adjacent arm 16s, this allows support member to return in a direction toward the untilted configuration shown in FIG. 5. It is further noted that shear driver 38 could alternatively be activated by a cam mechanism or a cable assembly attached to arm 20. Alternatively to use of lever as shear driver 38, a wedge may be used to drive the two support members 32, 34 apart on one side. The wedge can be driven by a shaft or screw mechanism.

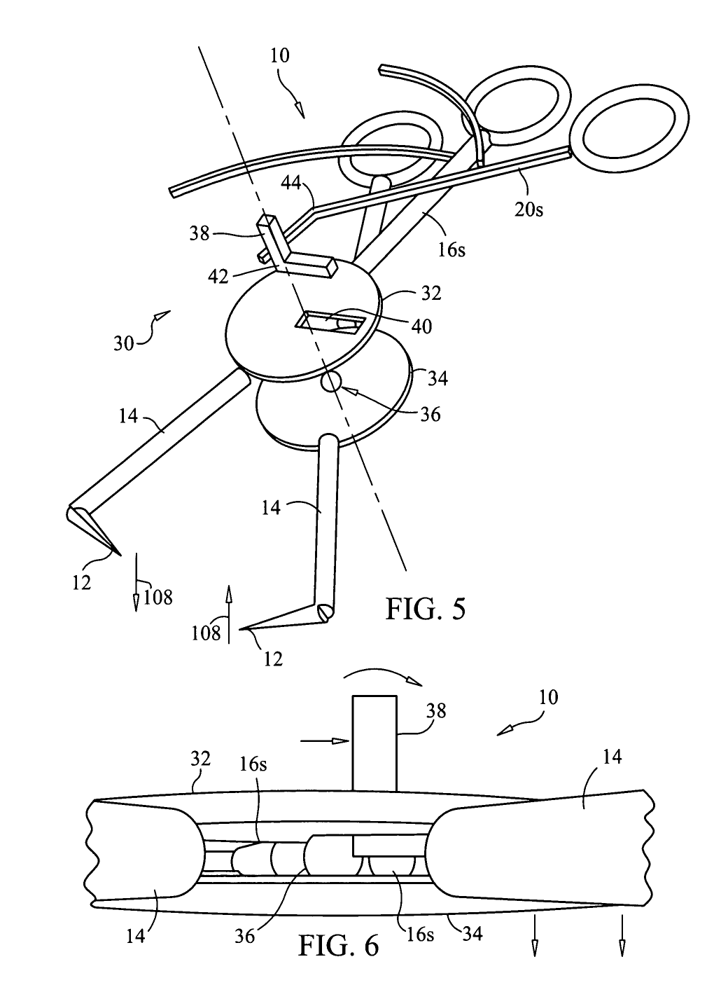

FIG. 6 is a partial, enlarged side view of instrument 10 showing the assembled components of the intermediate portion. Application of force against shear driver 38 by the distal end portion of shaft 20s in the direction indicated by the arrow causes rotation of shear driver 38 in the direction shown by the rotational arrow, such that the internally contained portion of shear drive 38 drives against support member 34 causing it to tilt about swivel bearing 36 in the same rotational direction as the rotational direction of shear driver 38. This cause a shearing action by driving the distal tip extending from the shaft 14 on the right side of FIG. 6 downward relative to the distal tip extending from the shaft 14 on the left side of FIG. 6.

When using instrument 10 to clamp together tissue pieces, such as fractured bone pieces, or other tissues to be clamped, distal tips 12 are contacted to the tissue pieces on surfaces opposite the surfaces of the tissue pieces to be joined. Distal tips 12 may be pointed or sharpened to facilitate engagement of the tips 12 with the tissue, and prevent slippage between the tips 12 and the tissue during clamping. Pointed or sharpened tips 12 can be particularly useful when the tissue pieces to be clamped are bone.

Compression actuators 16 can next be operated by the user to begin driving the tissue pieces together, by the movement of tips 12 toward one another. This action can continue until a the fractured surfaces contact one another, at which time, shear actuator 20 can be operated by the user to fine tune the alignment of the pieces, should any misalignment have occurred due to shear forces generated during the application of compression force by compression actuator 16. Once the tissue pieces have been accurately aligned by adjustment by the shear actuator 20, the compression actuator 16 can be further actuated to apply additional compressive force to securely clamp the pieces together. After each actuation of the compressive actuators 16, the relative positions of the actuators are locked by locking mechanism 18. After each actuator of shear actuator 20, the position of shear actuator 20 relative to the adjacent compression actuator shaft 16s is locked by locking mechanism 28.

Alternatively, compression actuators 16 and shear actuator 20 may be operated simultaneously, to adjust the tracking of the pieces as they are being joined together under compression, with shear adjustments being made as necessary according to feedback provided by visualization by the user of the relative positions of the pieces as they are being driven together.

Further alternatively, compression and shear may be alternatively and iteratively adjusted to effect an accurate fitting together of the pieces. For example, as the pieces are brought closer together by application of compressive forces, the user may note that the pieces are becoming misaligned. At that time, the relative positions of the tips under compression are maintained by locking mechanism 18 and shear actuator 20 is actuated to apply shear force to correct the alignment of the pieces. The relative positions of the tips 12 under shear are maintained by locking mechanism 28 and then additional compressive force can be applied via actuators 16 to being the pieces closer together. If this causes another misalignment, then additional shear force can be applied, and this process can continue iteratively until the pieces have been brought into contact under accurate alignment and with sufficient clamping force.

Preferably, the compression and shear forces are applied sequentially to maintain alignment of the pieces as they are driven together.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A clamping instrument comprising:
a distal portion having distal tips configured to be driven to provide clamping force to multiple tissue pieces;
a proximal portion including a compression actuator configured for operation by a user to drive said distal tips to apply compression force to the tissue pieces, and a shear actuator configured for operation by the user to drive said distal tips to apply shear force to the tissue pieces; and
an intermediate portion interconnecting said proximal and distal portions.

2. The instrument of claim 1, further comprising a relative position locking mechanism configured to temporarily maintain said distal tips in constant relative positions to apply a constant amount of compression.

3. The instrument of claim 1, further comprising a relative position locking mechanism configured to temporarily maintain said distal tips in constant relative positions to apply a constant amount of shear.

4. The instrument of claim 1, wherein said instrument comprises a bone clamp.

5. The instrument of claim 4, wherein the tissue pieces are fractured pieces of a bone.

6. The instrument of claim 1, wherein said distal portion comprises a pair of elongate shafts, each provided with one of said distal tips at a distal end thereof.

7. The instrument of claim 1, wherein said distal tips are directed inwardly toward one another.

8. The instrument of claim 1, wherein said intermediate portion comprises first and second supports, a first of said distal tips being connected to said first support and a second of said distal tips being connected to said second support, said shear actuator being operable to drive a relative tilting action between said first and second supports to generate said shear force in said distal tips.

9. The instrument of claim 1, wherein said intermediate portion comprises first and second supports, a first of said distal tips being connected to said first support and a second of said distal tips being connected to said second support, said compression actuator being operable to drive a relative rotation action between said first and second supports to generate said compression force in said distal tips by driving said distal tips toward one another.

10. The instrument of claim 1, wherein said intermediate portion comprises first and second supports, said first and second supports being connected via a joint that permits relative rotation between said first and second supports and relative tilting between said first and second supports.

11. The instrument of claim wherein said joint comprises a swivel bearing.

12. The instrument of claim 8, wherein said intermediate portion comprises a shear driver pivotally fixed to said intermediate portion and configured to provide a driving force against one of said first and second supports, when driven by said shear actuator, to tilt one of said first and second supports relative to the other of said first and second supports.

13. A method of joining tissues pieces together comprising the steps of:
applying first and second force applicators to first and second surfaces of first and second tissue pieces, wherein the first and second surfaces oppose third and fourth surfaces of the first and second tissue pieces to be joined, respectively;
applying compressive force, via said first and second force applicators, to drive the first and second pieces together; and
applying shear force, via said first and second force applicators, to accurately align the third and fourth surfaces, so that the third and fourth surfaces are contacted to one another in a desired fit pattern.

14. The method of claim 13, wherein the first and second force applicators are distal tips of a single clamping instrument.

15. The method of claim 13, wherein the tissue pieces are bone pieces, and the first and second force applicators are portions of a single bone clamping instrument.

16. The method of claim 13, wherein said applying compressive force is performed first, followed by fine tuning of alignment of the pieces by said applying shear forces.

17. The method of claim 13, wherein said applying compressive force and said applying shear force are applied simultaneously.

18. The method of claim 13, wherein said applying compressive force and said applying shear force are alternatively and iteratively adjusted to accurately align the tissue pieces and clamp them together to contact said third and fourth surfaces together in accurate alignment.

19. A method of accurately clamping fractured bone pieces together with a single bone clamp, said method comprising the steps of:
applying first and second distal tips of the bone clamp to first and second surfaces of first and second bone pieces, wherein the first and second surfaces oppose third and fourth fracture surfaces of the first and second bone pieces to be joined, respectively;
applying compressive force, via said first and second distal tips, to drive the first and second pieces together; and
applying shear force, via said first and second distal tips, to accurately align the third and fourth fracture surfaces, so that the third and fourth fracture surfaces are contacted to one another in a desired fit pattern.

20. The method of claim 19, wherein said applying compressive force and said applying shear force are independently controllable via a compression actuator and a shear actuator, respectively, provided in a proximal portion of the bone clamp.
